(19) United States
(12) Patent Application Publication
(10) Pub. No.: US 2025/0311951 A1
(43) Pub. Date: Oct. 9, 2025

(54) Blood Collection Tube Holder with Slidable Needle Safety Device

(71) Applicant: Retractable Technologies, Inc., Little Elm, TX (US)

(72) Inventors: Thomas J. Shaw, Frisco, TX (US); Benjamin John Lardas, Denton, TX (US)

(21) Appl. No.: 19/092,719

(22) Filed: Mar. 27, 2025

Related U.S. Application Data
(60) Provisional application No. 63/574,060, filed on Apr. 3, 2024.

(51) Int. Cl. A61B 5/15; A61B 5/153

(52) U.S. Cl. CPC A61B 5/150641; A61B 5/153

(57) ABSTRACT

Systems and methods are disclosed that include providing a blood collection tube with a unitarily molded barrel having a cylindrical body portion that defines a barrel cavity, a slot extending longitudinally along the cylindrical body portion, and opposing rails that extend longitudinally along edges of the cylindrical body portion formed by the slot. A dual-ended needle is retained within the barrel. A needle safety device having a pair of channels that receive the rails of the barrel is slidably engaged with the barrel between a first retracted position that exposes a venipuncture end of the needle for selective use of the blood collection tube holder and a second extended position that positions a needle tip shield of the needle safety device at least partially around the venipuncture end of the needle to protect a user from accidental needle sticks after use of the blood collection tube holder.

### CROSS-REFERENCE TO RELATED APPLICATIONS

This application claims priority to U.S. Provisional Application No. 63/574,060, filed on Apr. 3, 2024 by Thomas J. Shaw et al., and entitled “Blood Collection Tube Holder with Slidable Needle Safety Device,” the disclosure of which is incorporated by reference in its entirety for all purposes.

### BACKGROUND OF THE INVENTION

Patients who receive medical treatment or a medical diagnosis often require a blood draw for testing purposes. Blood collection tubes are used to collect and capture the blood from the blood draw. Blood collection tubes are generally inserted into a blood collection tube holder that has two separate needles. The two separate needles generally include a venipuncture needle pointing forwardly for intravenous insertion into the patient and a discharge needle pointing rearwardly and encapsulated within the blood collection tube holder. When a blood collection tube is inserted into the blood collection tube holder, the discharge needle punctures and penetrates an elastomeric stopper disposed on a forward end of the blood collection tube, such that blood from the patient can be captured from the patient through the venipuncture needle and pass through the discharge needle into the blood collection tube. Following collection of the blood sample, the blood collection tube may be removed from the blood collection tube holder. A hinged cap provided on the back of the blood collection tube holder may be closed by the medical professional performing the blood draw to prevent any blood or other bodily fluids inside the blood collection tube holder from leaking out.

Recent improvements include providing the blood collection tube holder with a retractable needle, where selectively activation of an actuator retracts the separate needles collectively into a needle retraction chamber coupled to the blood collection tube holder. Other recent improvements include providing the blood collection tube holder with separate venipuncture and discharge needles, where a needle retraction chamber for the venipuncture needle disposed adjacent to the blood collection tube holder, either as a separate chamber that is made integrally with the blood collection tube holder or as part of a frontal attachment to the blood collection tube holder, where lateral movement of an actuator repositions the venipuncture needle out of alignment with the rearwardly facing discharge needle and into alignment with the needle retraction chamber. Notwithstanding the improvements achieved through use of the aforementioned selectively retractable needles in blood collection tube holders, these blood collection tube holders are complex and often cost significantly more than traditional blood collection tube holders without retractable needle mechanisms. Accordingly, the medical industry continues to demand advances in blood collection tube holder technology that is both cost efficient and increases safety for the user.

### SUMMARY

In some aspects, the embodiments and techniques described herein relate to a blood collection tube holder, including: a unitarily molded barrel including a cylindrical body portion that defines a barrel cavity, a nose end extending from the cylindrical body portion and including an integral needle holder, a rear flange disposed around a rear end of the barrel cavity, a slot extending longitudinally along the cylindrical body portion from the nose end to the rear flange, and opposing rails that extend longitudinally along edges of the cylindrical body portion formed by the slot; a needle disposed within the needle holder and including a venipuncture end extending forwardly from the nose end of the barrel and a fluid discharge end extending rearwardly within the barrel cavity of the barrel; and a needle safety device including an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes the venipuncture end of the needle for selective use of the blood collection tube holder and a second extended position that positions the needle tip shield at least partially around the venipuncture end of the needle to protect a user from accidental needle sticks after use of the blood collection tube holder.

In some aspects, the embodiments and techniques described herein relate to a method of administering a blood draw, including: providing a blood collection tube holder including: a unitarily molded barrel including a cylindrical body portion that defines a barrel cavity, a nose end extending from the cylindrical body portion and including an integral needle holder, a rear flange disposed around a rear end of the barrel cavity, a slot extending longitudinally along the cylindrical body portion from the nose end to the rear flange, and opposing rails that extend longitudinally along edges of the cylindrical body portion formed by the slot; a needle disposed within the needle holder and including a venipuncture end extending forwardly from the nose end of the barrel and a discharge end extending rearwardly within the barrel cavity of the barrel; and a needle safety device including an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes the venipuncture end of the needle for selective use of the blood collection tube holder and a second extended position that positions the needle tip shield at least partially around the venipuncture end of the needle to protect a user from accidental needle sticks after use of the blood collection tube holder; inserting a blood collection tube into the blood collection tube holder; performing a blood draw on a patient; and activating the needle safety device to prevent unintended needle sticks or inadvertent contact with the venipuncture end of the needle upon completion of the blood draw.

### BRIEF DESCRIPTION OF THE DRAWINGS

For a more complete understanding of the present disclosure and so that the manner in which the features and advantages of the embodiments can be understood in more detail, reference is now made to the following brief description, taken in connection with the accompanying drawings and detailed description.

FIG. 1 shows an oblique view of a blood collection tube holder according to an embodiment of the disclosure.

FIG. 2 shows an oblique exploded view of the blood collection tube holder according to an embodiment of the disclosure.

FIG. 3 shows a detailed partial cross-sectional front view of a barrel and a needle safety device of the blood collection tube holder according to an embodiment of the disclosure.

FIG. 4 shows an orthogonal cross-sectional side view of the blood collection tube holder prepared for use according to an embodiment of the disclosure.

FIG. 5 shows an orthogonal side view of the blood collection tube holder after use according to an embodiment of the disclosure.

FIG. 6 shows an oblique view of a blood collection tube holder according to another embodiment of the disclosure.

FIG. 7 shows an oblique exploded view of the blood collection tube holder according to another embodiment of the disclosure.

FIG. 8 shows a detailed partial cross-sectional front view of a barrel and a needle safety device of the blood collection tube holder according to another embodiment of the disclosure.

FIG. 9 shows a flowchart of a method of administering a blood draw with the blood collection tube holder according to an embodiment of the disclosure.

The use of the same reference symbols in different drawings indicates similar or identical items.

### DETAILED DESCRIPTION

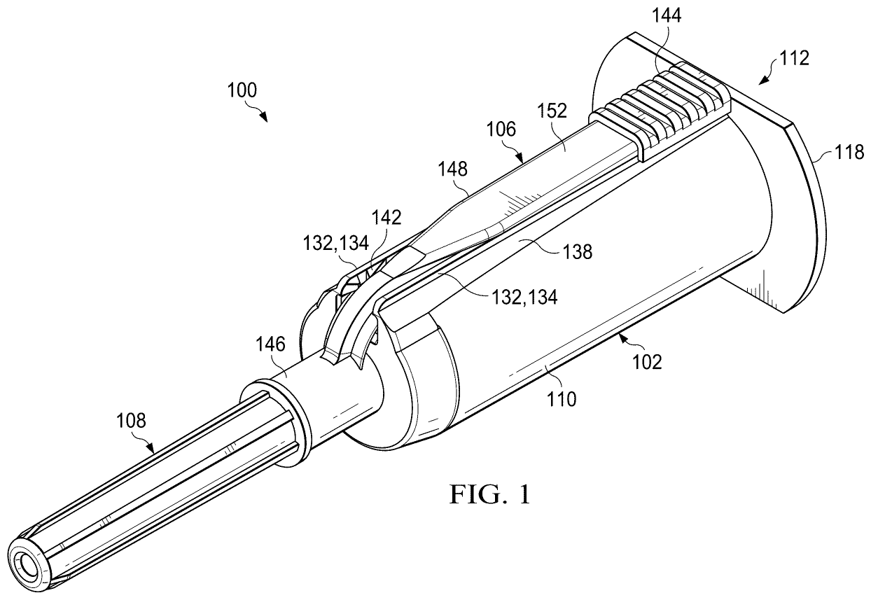

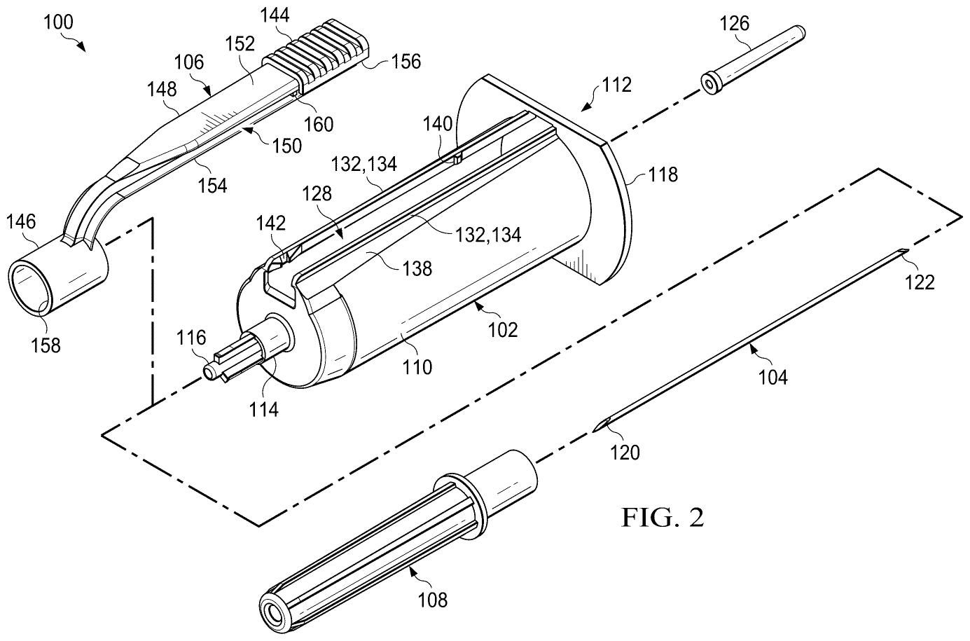

Referring to FIGS. 1 and 2, an oblique view and an oblique exploded view of a blood collection tube holder 100 are shown according to an embodiment of the disclosure. Blood collection tube holder 100 may preferably comprise a unitarily molded barrel 102 preferably molded from a medical grade polymeric material, a needle 104, and a needle safety device 106. In some embodiments, blood collection tube holder 100 may also comprise a needle cap 108 covering the needle 104 and at least a portion of the barrel 102 prior to use. Barrel 102 may comprise a cylindrical body portion 110 that defines a barrel cavity 112 that is open rearwardly, a nose end 114 extending from the cylindrical body portion 110 and comprising an integral needle holder 116, and a rear flange 118 disposed around a rear end of the barrel cavity 112 for selective manipulation of the blood collection tube holder 100.

Needle 104 may preferably be carried by and secured within the needle holder 116 of the barrel 102. In some embodiments, needle 104 may be secured within the needle holder 116 via friction and/or an interference fit. In some embodiments, medical grade adhesive may be applied to secure the needle 104 within the needle holder 116. In alternative embodiments, needle holder 116 may comprise a separate component that is threadably attachable and removable from the nose end 114 of the barrel 102 to secure the needle 104 within the barrel 102.

In a preferred embodiment, needle 104 may comprise a single needle that comprises a forwardly extending venipuncture end 120 and rearwardly extending fluid discharge end 122. Venipuncture end 120 preferably extends forwardly from the nose end 114 of the barrel 102 for intravenous insertion into a patient, while discharge end 122 preferably extends rearwardly within the barrel cavity 112 of the barrel for insertion into a blood collection tube (not pictured) to capture drawn blood or other fluids from the patient. However, in some embodiments, needle 104 may comprise two separate needle components in fluid communication through the needle holder 116, with a first needle comprising a forwardly extending venipuncture needle and the second needle being a rearwardly extending fluid discharge needle.

The needle holder 116 of the barrel 102 may comprise a rearwardly extending fitting 124 configured to receive and retain an elastomeric sheath 126. Elastomeric sheath 126 may be configured to cover the fluid discharge end 122 of the needle 104 when no blood collection is inserted into the barrel cavity 112 and to collapse forwardly around the fluid discharge end 122 of the needle 104 when a blood collection tube is inserted into the barrel cavity 112 to allow fluid discharge end 122 to penetrate an elastomeric stopper in the blood collection tube to establish fluid communication between the needle 104 and the blood collection tube into which the drawn blood is discharged.

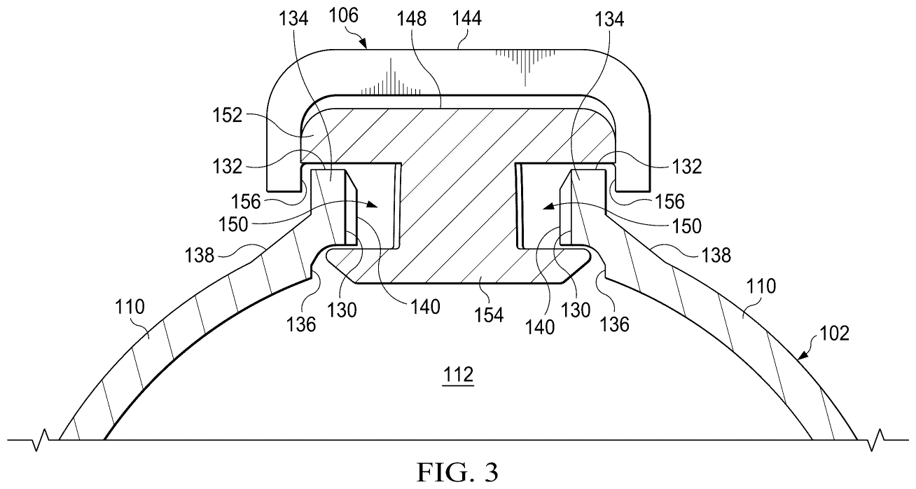

Referring to FIGS. 1 and 2, in conjunction with FIG. 3 that shows a detailed partial cross-sectional front view of barrel 102 and needle safety device 106 according to an embodiment of the disclosure, barrel 102 and needle safety device 106 may be cooperatively slidably engaged to allow selective actuation of the needle safety device 106 to protect users from accidental needle sticks after use of the blood collection tube holder 100. To provide the cooperatively slidably engagement, barrel 102 may be formed with a slot 128 extending longitudinally along the cylindrical body portion 110 of the barrel 102 from the nose end 114 of the barrel 102 to the rear flange 118 of the barrel 102.

Opposing edges 130 of the cylindrical body portion 110 formed along the slot 128 have raised portions 132 that extend longitudinally along the slot 128 and are raised outwardly from the cylindrical body portion 110 of the barrel 102. Raised portions 132 may extend in a parallel manner along the cylindrical body portion to form raised rails 134 along which the needle safety device 106 can slide. In some embodiments, rails 134 may extend to and be integral with the rear flange 118 to provide structural integrity to the cylindrical body portion 110 of the barrel 102. In some embodiments, rails 134 may extend along the entire length of cylindrical body portion 110. In other embodiments, rails 134 may extend less than the entire length of the cylindrical body portion 110. In some embodiments, edges 130 may comprise radiused undercut portions 136 on an inside of the cylindrical body portion 110 of the barrel 102 that may provide clearance for and/or selectively receive portions of the needle safety device 106. In some embodiments, a bevel or chamfer 138 may be formed between raised portions 132 and cylindrical body portion 110 to increase the structural integrity of the slot 128 and/or the barrel 102.

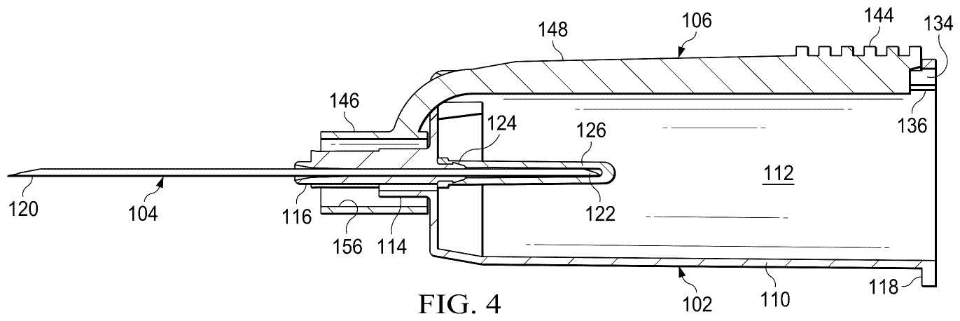

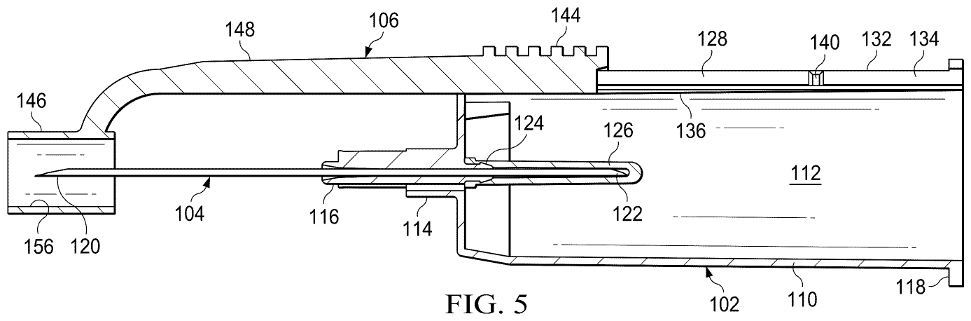

Raised portions 132 may also comprise a rear stop 140 and a slide stop 142 disposed forwardly from the rear stop 140. Rear stop 140 and slide stop 142 may preferably extend inwardly from the raised portions 132 and/or rails 134 and reduce a width between the raised portions 132 and/or rails 134. Rear stop 140 may be configured to engage the needle safety device 106 and retain the needle safety device 106 in a first retracted position (shown in FIG. 4) to expose the venipuncture end 120 of the needle 104 for selective use of the blood collection tube holder 100. Slide stop 142 may be configured to engage the needle safety device 106 and retain the needle safety device 106 in a second extended position (shown in FIG. 5) that is forward of the first retracted position to cover up the venipuncture end 120 of the needle 104 after use of the blood collection tube holder 100 to protect a user or other from accidental “sticks” from an otherwise exposed needle of a traditional blood collection tube holder not having the needle safety device 106.

Needle safety device 106 may be unitarily molded from a polymeric material. In some embodiments, needle safety device 106 may be formed from a clear or transparent material that allows viewing through the needle safety device 106 and into the barrel cavity 112 of the blood tube collection holder 100 to view the presence of or amount of a fluid drawn into a blood collection tube and/or watch for blood flashback, which may be used to confirm that the needle 104 has been inserted intravenously into a patient.

Needle safety device 106 may comprise an activation surface 144 used to slide the needle safety device 106 between the first retracted position and the second extended position, a needle tip shield 146, and a main body portion 148 connecting the activation surface 144 and the needle tip shield 146. Needle safety device 106 may also comprise a pair of channels 150 disposed along and formed in main body portion 148. Channels 150 may be formed in main body portion 148 of needle safety device 106 such that an upper portion 152 of the main body portion 148 extends over the raised portions 132 and/or rails 134 of the barrel 102, and such that a lower portion 154 of the main body portion 148 extends below the raised portions 132 and/or rails 134 of the barrel 102 and extends into the radiused undercut portions 136 of the barrel 102. In some embodiments, extended portions 156 of activation surface 144 of needle safety device 106 may vertically overlap over channels 150 to lock raised portions 132 and/or rails 134 of the barrel 102 within the channels 150. In other embodiments, upper portion 152 of main body portion 148 may vertically overlap over channels 150 to lock raised portions 132 and/or rails 134 of the barrel 102 within the channels 150.

When assembled, raised portions 132 and/or rails 134 of the barrel 102 formed in the cylindrical body portion of the barrel 102 may be received within channels 150 of the needle safety device 106 to provide a smooth, slidable interface between barrel 102 and needle safety device 106. Extended portions 156 of activation surface 144 and/or upper portion 152 of main body portion 148 may vertically overlap the channels 150 to lock raised portions 132 and/or rails 134 of the barrel 102 within the channels 150. In this manner, the lower portion 154 of the main body portion 148 of the needle safety device 106 may form a portion of the barrel 102 when the blood collection tube holder 100 is in use and allows such portion of the barrel 102 to slide forward when use of the blood collection tube holder 100 is complete. By having the needle safety device 106 form a portion of the barrel 102 when in use, and subsequently sliding the needle safety device 106 forward along the barrel 102 from the first retracted position and the second extended position, material used in manufacturing blood collection tube holder 100 may be drastically reduced by the needle safety device 106 serving this dual purpose.

Referring to FIG. 4, an orthogonal cross-sectional side view of the blood collection tube holder 100 prepared for use is shown according to an embodiment of the disclosure. Prior to insertion of a blood collection tube (not shown) into barrel cavity 112 and use of blood collection tube holder 100 for collecting blood from a patient, needle cap 108 may be disposed within an inner bore 158 of needle tip shield 146 of the needle safety device 106 and around the nose end 114 of the barrel 102 to cover the venipuncture end 120 of the needle 104. Elastomeric sheath 126 may cover fluid discharge end 122 of the needle 104 to shield fluid discharge end 122 from inadvertent contamination. With the needle safety device 106 secured in the first retracted position by rear stop 140 engaging a rib 160 disposed in each channel 150 of the needle safety device, the needle cap 108 may be removed from the blood collection tube holder 100 to prepare the blood collection tube holder 100 for use. When a blood collection tube is inserted into barrel cavity 112 of barrel 102, elastomeric sheath 126 may collapse forwardly around the needle 104, thereby allowing the fluid discharge end 122 of the needle 104 to penetrate an elastomeric stopper in the blood collection tube to establish fluid communication between the venipuncture end 120 of the needle 104 and the inside of the blood collection tube into which the drawn blood is discharged.

Referring to FIG. 5, an orthogonal side view and an orthogonal cross-sectional side view of the blood collection tube holder 100 after use are shown according to an embodiment of the disclosure. After administration of the blood draw with the blood collection tube holder 100, the needle safety device 106 may be activated forwardly by pressing on the activation surface 144. As the needle safety device 106 slides forwardly, slide stop 142 may engage ribs 160 of the needle safety device 106 to retain the needle safety device 106 in the second extended position. In the second extended position, the venipuncture end 120 of the needle 104 may be disposed within the inner bore 158 of the needle tip shield 146 of the needle safety device 106, thereby circumferentially surrounding the venipuncture end 120 of the needle 104. Once engaged, slide stop 142 effectively prevents the needle safety device 106 from being slid backwards to uncover the venipuncture end 120 of the needle 104, thereby preventing unintended needle sticks or inadvertent contact with the needle 104.

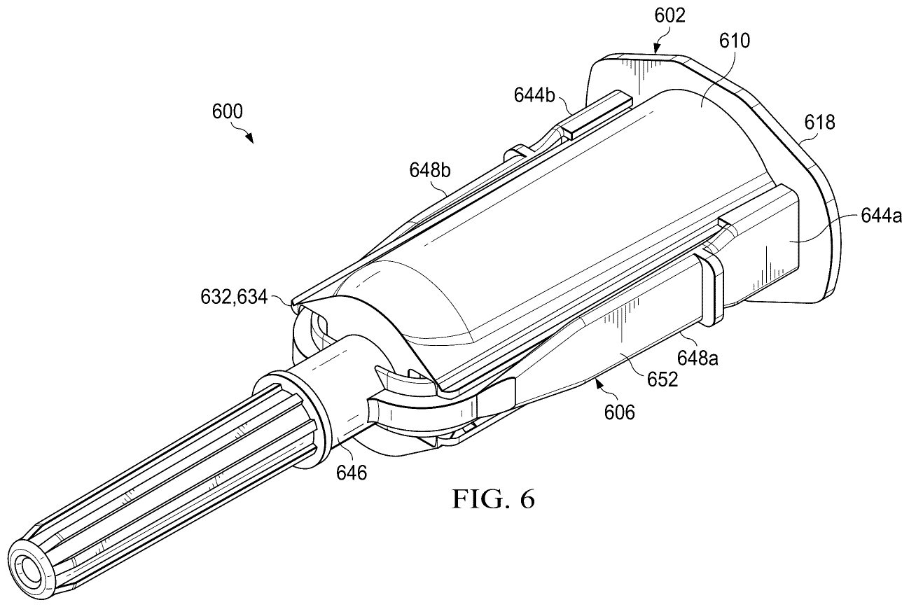

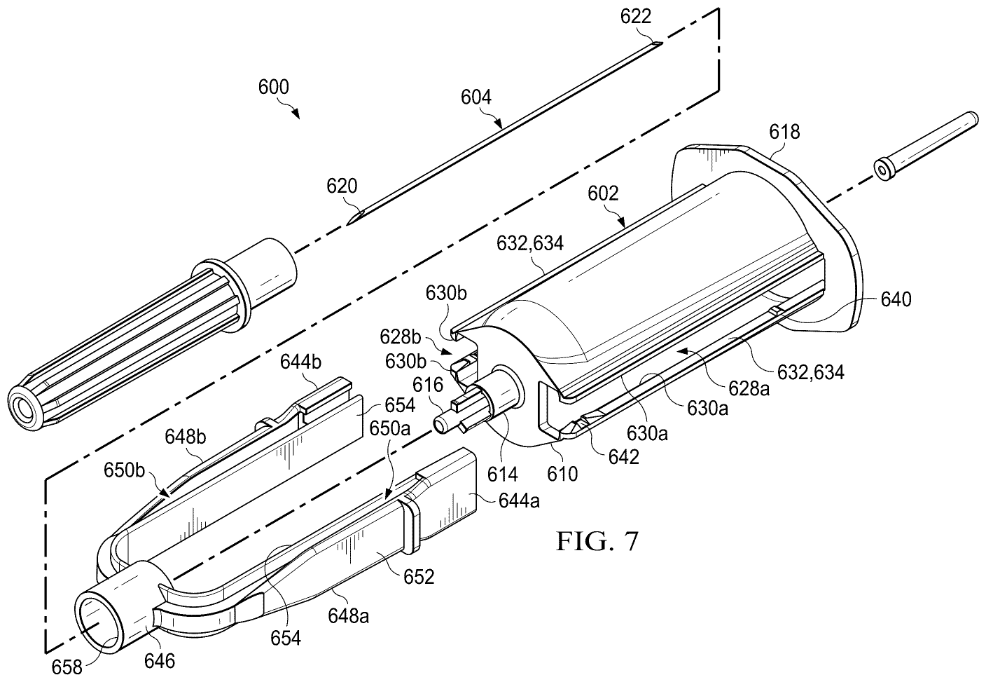

Referring to FIGS. 6 and 7, an oblique view, an oblique exploded view, and a detailed partial cross-sectional side view of a blood collection tube holder 600 are shown according to another embodiment of the disclosure. Blood collection tube holder 600 may generally be substantially similar to blood collection tube holder 100 and comprise a unitarily molded barrel 602 preferably molded from a medical grade polymeric material, a needle 604 secured within a needle holder 616 and comprising a single needle that comprises a forwardly extending venipuncture end 620 and rearwardly extending fluid discharge end 622, and a needle safety device 606. However, needle safety device 606 comprises a dual arm needle safety device.

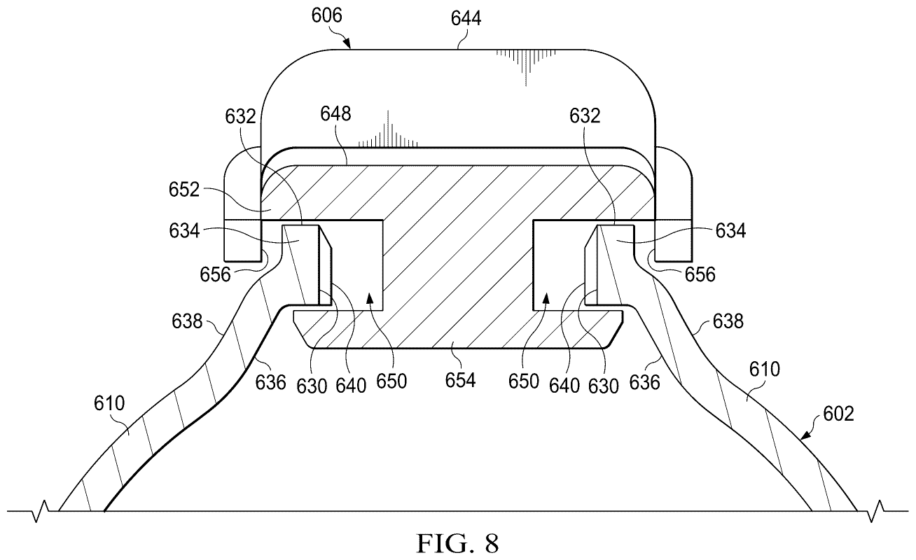

Referring to FIGS. 6 and 7, in conjunction with FIG. 8 that shows a detailed partial cross-sectional front view of barrel 602 and needle safety device 606, barrel 602 and needle safety device 606 may be cooperatively slidably engaged, similarly to blood collection tube holder 100, to allow selective actuation of the needle safety device 606 to protect users from accidental needle sticks after use of the blood collection tube holder 600 in a similar manner as blood collection tube holder 100. To provide the cooperatively slidably engagement, barrel 602 may be formed with opposing slots 628a, 628b extending longitudinally along a cylindrical body portion 610 of the barrel 602 from a nose end 614 of the barrel 602 to a rear flange 618 of the barrel 602.

Opposing edges 630a, 630b of the cylindrical body portion 610 formed along the slots 628a, 628b have raised portions 632 that extend longitudinally along the slots 628a, 628b and are raised outwardly from the cylindrical body portion 610 of the barrel 602. Raised portions 632 may extend in a parallel manner along the cylindrical body portion to form raised rails 634 along which the needle safety device 606 can slide. In some embodiments, rails 634 may extend to and be integral with the rear flange 618 to provide structural integrity to the cylindrical body portion 610 of the barrel 602. In some embodiments, rails 634 may extend along the entire length of cylindrical body portion 610. In other embodiments, rails 634 may extend less than the entire length of the cylindrical body portion 610. In some embodiments, edges 630 may comprise radiused undercut portions 636 on an inside of the cylindrical body portion 610 of the barrel 602 that may provide clearance for and/or selectively receive portions of the needle safety device 606. In some embodiments, a bevel or chamfer 638 may be formed between raised portions 632 and cylindrical body portion 610 to increase the structural integrity of the slot 628 and/or the barrel 602.

Raised portions 632 may also comprise a rear stop 640 and a slide stop 642 disposed forwardly from the rear stop 640. Rear stop 640 and slide stop 642 may preferably extend inwardly from the raised portions 632 and/or rails 634 and reduce a width between the raised portions 632 and/or rails 634. Rear stop 640 may be configured to engage the needle safety device 606 and retain the needle safety device 606 in a first retracted position to expose the venipuncture end 620 of the needle 604 for selective use of the blood collection tube holder 600. Slide stop 642 may be configured to engage the needle safety device 606 and retain the needle safety device 606 in a second extended position that is forward of the first retracted position to cover up the venipuncture end 620 of the needle 604 after use of the blood collection tube holder 600 to protect a user or other from accidental “sticks” from an otherwise exposed needle of a traditional blood collection tube holder not having the needle safety device 606.

Needle safety device 606 may comprise a dual arm needle safety device and includes opposing activation surfaces 644a, 644b used to slide the needle safety device 606 between the first retracted position and the second extended position, a needle tip shield 646, and main body portions 648a, 648b connecting the activation surfaces 644a, 644b and the needle tip shield 646. Needle safety device 606 may also comprise a pair of channels 650a, 650b disposed along and formed in main body portions 648a, 648b. Channels 650a, 650b may be formed in main body portions 648a, 648b of needle safety device 606 such that an upper portion 652 of the main body portions 648a, 648b extends over the raised portions 632 and/or rails 634 of the barrel 602, and such that a lower portion 654 of the main body portions 648a, 648b extends below the raised portions 632 and/or rails 634 of the barrel 602 and extends into the radiused undercut portions 636 of the barrel 602. In some embodiments, extended portions 656 of activation surfaces 644a, 644b of needle safety device 606 may vertically overlap over channels 650a, 650b to lock raised portions 632 and/or rails 634 of the barrel 602 within the channels 650a, 650b. In other embodiments, upper portion 652 of main body portions 648a, 648b may vertically overlap over channels 650a, 650b to lock raised portions 632 and/or rails 634 of the barrel 602 within the channels 650a, 650b.

When assembled, raised portions 632 and/or rails 634 of the barrel 602 formed in the cylindrical body portion of the barrel 602 may be received within channels 650 of the needle safety device 606 to provide a smooth, dually slidable interface between barrel 602 and needle safety device 606. Extended portions 656 of activation surface 644 and/or upper portion 652 of main body portions 648a, 648b may vertically overlap the channels 650a, 650b to lock raised portions 632 and/or rails 634 of the barrel 602 within the channels 650a, 650b. In this manner, the lower portion 654 of the main body portions 648a, 648b of the needle safety device 606 may form a portion of the barrel 602 when the blood collection tube holder 600 is in use and allows such portion of the barrel 602 to slide forward when use of the blood collection tube holder 600 is complete. By having the needle safety device 606 form a portion of the barrel 602 when in use, and subsequently sliding the needle safety device 606 forward along the barrel 602 from the first retracted position and the second extended position, material used in manufacturing blood collection tube holder 600 may be drastically reduced by the needle safety device 606 serving this dual purpose.

Use of the blood collection tube holder 600 may also be substantially similar to blood collection tube holder 100. Prior to insertion of a blood collection tube (not shown) into barrel 602 and use of blood collection tube holder 600 for collecting blood from a patient, the needle safety device 606 secured in the first retracted position by rear stop 640 engaging a rib disposed in each channel 650a, 650b of the needle safety device. When a blood collection tube is inserted into barrel cavity 612 of barrel 602, the fluid discharge end 622 of the needle 604 may penetrate an elastomeric stopper in the blood collection tube to establish fluid communication between the venipuncture end 620 of the needle 604 and the inside of the blood collection tube into which the drawn blood is discharged. After administration of the blood draw with the blood collection tube holder 600, the needle safety device 606 may be activated forwardly by pressing on the activation surface 644. As the needle safety device 606 slides forwardly, slide stop 642 may engage ribs of the needle safety device 606 to retain the needle safety device 606 in the second extended position. In the second extended position, the venipuncture end 620 of the needle 604 may be disposed within the inner bore 658 of the needle tip shield 646 of the needle safety device 606, thereby circumferentially surrounding the venipuncture end 620 of the needle 604. Once engaged, slide stop 642 effectively prevents the needle safety device 606 from being slid backwards to uncover the venipuncture end 620 of the needle 604, thereby preventing unintended needle sticks or inadvertent contact with the needle 604.

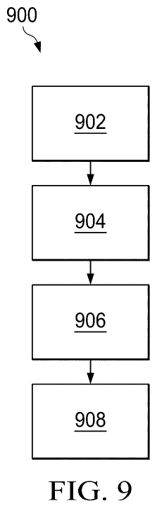

FIG. 9 shows a flowchart of a method 900 of administering a blood draw with a blood collection tube holder 100, 600 according to an embodiment of the disclosure. The method 900 may begin at block 902 by providing a blood collection tube holder 100, 600 as disclosed herein. In some embodiments, the blood collection tube holder 100, 600 may comprise a unitarily molded barrel 102, 602, a needle 104, 604, and a needle safety device 106, 606. The method 900 may continue at block 904 by inserting a blood collection tube into the blood collection tube holder 100, 600. The method 900 may continue at block 906 by performing a blood draw on a patient. In some embodiments, performing the blood draw on a patient may comprise inserting the venipuncture end 120, 620 of needle 104, 604 into a patient, collecting blood from the patient in the blood collection tube, and removing the venipuncture end 120, 620 of the needle 104, 604 from the patient. In some embodiments, performing the blood draw on a patient may also comprise removing the blood collection tube from the blood collection tube holder 100, 600. The method 900 may continue at block 908 by activating the needle safety device 106, 606 to prevent unintended needle sticks or inadvertent contact with venipuncture end 120, 620 of needle 104, 604. In some embodiments, preventing unintended needle sticks or inadvertent contact with venipuncture end 120, 620 of needle 104, 604 may be accomplished by sliding the needle safety device 106, 606 from a first retracted position to a second extended position to cause a needle tip shield 146, 646 of the needle safety device 106, 606 to circumferentially surround venipuncture end 120, 620 of needle 104, 604 and retaining the needle safety device 106, 606 in the second extended position by engagement of slide stop 142, 642 disposed on rails 134, 634 of the barrel 102, 602 with ribs 160, 660 disposed in channels 150, 650 of the needle safety device 106, 606.

While embodiments of a blood collection tube holder 100, 600 are disclosed, the systems and methods may be equally applicable to any container, holder, or other apparatus that includes a safety device that slides with respect to the barrel and that forms a portion of the barrel. These embodiments may be applicable to both medical devices and non-medical devices.

It will be appreciated that embodiments of a blood collection tube holder 100, 600 and/or a method 900 of administering a blood draw with a blood collection tube holder 100, 600 may include one or more of the following embodiments:

Embodiment 1. A medical device, comprising: a unitarily molded barrel comprising a cylindrical body portion that defines a barrel cavity, a nose end extending from the cylindrical body portion and comprising an integral needle holder, a rear flange disposed around a rear end of the barrel cavity, a slot extending longitudinally along the cylindrical body portion from the nose end to the rear flange, and opposing rails that extend longitudinally along edges of the cylindrical body portion formed by the slot; a needle disposed within the needle holder and comprising a venipuncture end extending forwardly from the nose end of the barrel and a fluid discharge end extending rearwardly within the barrel cavity of the barrel; and a needle safety device comprising an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes the venipuncture end of the needle for selective use of the medical device and a second extended position that positions the needle tip shield at least partially around the venipuncture end of the needle to protect a user from accidental needle sticks after use of the medical device.

Embodiment 2. The medical device of embodiment 1, wherein the barrel is formed from a medical grade polymeric material.

Embodiment 3. The medical device of embodiment 1, wherein the needle safety device is formed from a medical grade polymeric material.

Embodiment 4. The medical device of embodiment 3, wherein the needle safety device is formed from a clear medical grade polymeric material that functions as a window to inspect the barrel cavity for blood flashback or a flow of fluid therethrough.

Embodiment 5. The medical device of embodiment 1, wherein the needle is secured within the needle holder via friction or an interference fit.

Embodiment 6. The medical device of embodiment 1, wherein the needle is secured within the needle holder via a medical grade adhesive.

Embodiment 7. The medical device of embodiment 1, wherein the needle holder is threadably attached to the barrel to secure the needle within the barrel.

Embodiment 8. The medical device of embodiment 1, wherein the venipuncture end of the needle is configured for intravenous insertion into a patient, and wherein the fluid discharge end extends rearwardly within the barrel cavity of the barrel for coupling to a fluid apparatus.

Embodiment 9. The medical device of embodiment 8, wherein the needle holder comprises a rearwardly extending fitting configured to receive an elastomeric sheath configured to cover the fluid discharge end of the needle when no fluid apparatus is inserted into the barrel cavity of the barrel and collapse forwardly around the fluid discharge end of the needle when a fluid apparatus is inserted into the barrel cavity to couple the fluid discharge end of the needle to the fluid apparatus to establish fluid communication between the venipuncture end of the needle and the fluid apparatus.

Embodiment 10. The medical device of embodiment 9, wherein the medical device comprises a blood collection tube holder.

Embodiment 11. The medical device of embodiment 10, wherein the fluid apparatus comprises a blood collection tube.

Embodiment 12. The medical device of embodiment 9, wherein the medical device comprises a blood collection tube holder.

Embodiment 13. The medical device of embodiment 12, wherein the fluid apparatus comprises a fluid delivery apparatus comprising flexible tubing, a flexible tubing coupling, or a combination thereof coupled to a fluid container containing a fluid configured to be delivered from the fluid container through the medical device.

Embodiment 14. The medical device of embodiment 1, wherein the rails extend in a parallel manner along the cylindrical body portion to form raised rails along which the needle safety device can slide.

Embodiment 15. The medical device of embodiment 14, wherein the rails extend to and are integral with the rear flange to provide structural integrity to the cylindrical body portion of the barrel.

Embodiment 16. The medical device of embodiment 1, wherein the channels are formed in main body portion of the needle safety device such that an upper portion of the main body portion extends over the rails of the barrel, and such that a lower portion of the main body portion extends below the rails of the barrel.

Embodiment 17. The medical device of embodiment 16, wherein the lower portion of the main body portion extends into radiused undercut portions formed on an inside of the cylindrical body portion of the barrel.

Embodiment 18. The medical device of embodiment 17, wherein the lower portion of the main body portion forms a portion of the barrel when the medical device is in use, and wherein the portion of the barrel formed by the lower portion of the main body portion of the needle safety device is selectively slidable after use of the medical device.

Embodiment 19. The medical device of embodiment 1, wherein extended portions of the activation surface of the needle safety device overlap the channels to lock the rails of the barrel within the channels.

Embodiment 20. The medical device of embodiment 1, wherein each of the rails comprise a rear stop and a slide stop disposed forwardly from the rear stop.

Embodiment 21. The medical device of embodiment 20, wherein the rear stops and slide stops extend inwardly from the rails.

Embodiment 22. The medical device of embodiment 21, wherein the rear stops are configured to engage ribs on the needle safety device to retain the needle safety device in the first retracted position to expose the venipuncture end of the needle for selective use of the medical device.

Embodiment 23. The medical device of embodiment 22, wherein the slide stops are configured to engage the ribs on the needle safety device and retain the needle safety device in the second extended position that is forward of the first retracted position to cover up the venipuncture end of the needle after use of the medical device to protect a user from accidental needle sticks after use of the medical device.

Embodiment 24. The medical device of embodiment 1, wherein the barrel comprises two opposing slots disposed on opposite sides of the barrel and extending longitudinally along the cylindrical body portion from the nose end to the rear flange, wherein each of the two slots comprise opposing rails that extend longitudinally along edges of the cylindrical body portion formed by each of the two slots.

Embodiment 25. The medical device of embodiment 24, wherein the needle safety device comprises a dual arm needle safety device that is slidably engaged with the two opposing slots disposed on opposite sides of the barrel.

Embodiment 26. The medical device of embodiment 25, wherein needle safety device comprises a plurality of main body portions extending from the needle tip shield and a pair of channels disposed along and formed in each of the main body portions, wherein the rails of each of the two opposing slots disposed on opposite sides of the barrel are received within the channels of the needle safety device to provide a slidable interface between the needle safety device and the barrel between the first retracted position and the second extended position.

Embodiment 27. A blood collection tube holder, comprising: a unitarily molded barrel comprising a cylindrical body portion that defines a barrel cavity, a nose end extending from the cylindrical body portion and comprising an integral needle holder, a rear flange disposed around a rear end of the barrel cavity, a slot extending longitudinally along the cylindrical body portion from the nose end to the rear flange, and opposing rails that extend longitudinally along edges of the cylindrical body portion formed by the slot; and a needle safety device comprising an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes a venipuncture end of a needle for selective use of the blood collection tube holder and a second extended position that positions the needle tip shield at least partially around the venipuncture end of the needle to protect a user from accidental needle sticks after use of the blood collection tube holder.

Embodiment 28. The blood collection tube holder of embodiment 27, wherein the barrel is formed from a medical grade polymeric material.

Embodiment 29. The blood collection tube holder of embodiment 28, wherein the needle safety device is formed from a medical grade polymeric material.

Embodiment 30. The blood collection tube holder of embodiment 29, wherein the needle safety device is formed from a clear medical grade polymeric material that functions as a window to inspect the barrel cavity for blood flashback or a flow of fluid therethrough.

Embodiment 31. The blood collection tube holder of embodiment 27, further comprising: a needle disposed within the needle holder and comprising a venipuncture end extending forwardly from the nose end of the barrel and a fluid discharge end extending rearwardly within the barrel cavity of the barrel.

Embodiment 32. The blood collection tube holder of embodiment 31, wherein the needle is secured within the needle holder via friction or an interference fit.

Embodiment 33. The blood collection tube holder of embodiment 31, wherein the needle is secured within the needle holder via a medical grade adhesive.

Embodiment 34. The blood collection tube holder of embodiment 31, wherein the needle holder is threadably attached to the barrel to secure the needle within the barrel.

Embodiment 35. The blood collection tube holder of embodiment 31, wherein the venipuncture end of the needle is configured for intravenous insertion into a patient, and wherein the fluid discharge end extends rearwardly within the barrel cavity of the barrel for insertion into a blood collection tube.

Embodiment 36. The blood collection tube holder of embodiment 35, wherein the needle holder comprises a rearwardly extending fitting configured to receive an elastomeric sheath configured to cover the fluid discharge end of the needle when no blood collection is inserted into the barrel cavity of the barrel and collapse forwardly around the fluid discharge end of the needle when a blood collection tube is inserted into the barrel cavity to penetrate the blood collection tube to establish fluid communication between the venipuncture end of the needle and the blood collection tube.

Embodiment 37. The blood collection tube holder of embodiment 27, wherein the rails extend in a parallel manner along the cylindrical body portion to form raised rails along which the needle safety device can slide.

Embodiment 38. The blood collection tube holder of embodiment 37, wherein the rails extend to and are integral with the rear flange to provide structural integrity to the cylindrical body portion of the barrel.

Embodiment 39. The blood collection tube holder of embodiment 27, wherein the channels are formed in main body portion of the needle safety device such that an upper portion of the main body portion extends over the rails of the barrel, and such that a lower portion of the main body portion extends below the rails of the barrel.

Embodiment 40. The blood collection tube holder of embodiment 39, wherein the lower portion of the main body portion extends into radiused undercut portions formed on an inside of the cylindrical body portion of the barrel.

Embodiment 41. The blood collection tube holder of embodiment 40, wherein the lower portion of the main body portion forms a portion of the barrel when the blood collection tube holder is in use, and wherein the portion of the barrel formed by the lower portion of the main body portion of the needle safety device is selectively slidable after use of the blood collection tube holder.

Embodiment 42. The blood collection tube holder of embodiment 27, wherein extended portions of the activation surface of the needle safety device overlap the channels to lock the rails of the barrel within the channels.

Embodiment 43. The blood collection tube holder of embodiment 27, wherein each of the rails comprise a rear stop and a slide stop disposed forwardly from the rear stop.

Embodiment 44. The blood collection tube holder of embodiment 43, wherein the rear stops and slide stops extend inwardly from the rails.

Embodiment 45. The blood collection tube holder of embodiment 44, wherein the rear stops are configured to engage ribs on the needle safety device to retain the needle safety device in the first retracted position to expose the venipuncture end of the needle for selective use of the blood collection tube holder.

Embodiment 46. The blood collection tube holder of embodiment 45, wherein the slide stops are configured to engage the ribs on the needle safety device and retain the needle safety device in the second extended position that is forward of the first retracted position to cover up the venipuncture end of the needle after use of the blood collection tube holder to protect a user from accidental needle sticks after use of the blood collection tube holder.

Embodiment 47. The blood collection tube holder of embodiment 27, wherein the needle safety device comprises a support arm extending from the needle tip shield and slidably engaged with the barrel.

Embodiment 48. The blood collection tube holder of embodiment 27, wherein the barrel comprises two opposing slots disposed on opposite sides of the barrel and extending longitudinally along the cylindrical body portion from the nose end to the rear flange, wherein each of the two slots comprise opposing rails that extend longitudinally along edges of the cylindrical body portion formed by each of the two slots.

Embodiment 49. The blood collection tube holder of embodiment 48, wherein the needle safety device comprises a dual arm needle safety device that is slidably engaged with the two opposing slots disposed on opposite sides of the barrel.

Embodiment 50. The blood collection tube holder of embodiment 49, wherein needle safety device comprises a plurality of main body portions extending from the needle tip shield and a pair of channels disposed along and formed in each of the main body portions, wherein the rails of each of the two opposing slots disposed on opposite sides of the barrel are received within the channels of the needle safety device to provide a slidable interface between the needle safety device and the barrel between the first retracted position and the second extended position.

Embodiment 51. A method of administering a blood draw, comprising: providing a blood collection tube holder comprising: a unitarily molded barrel comprising a cylindrical body portion that defines a barrel cavity, a nose end extending from the cylindrical body portion and comprising an integral needle holder, a rear flange disposed around a rear end of the barrel cavity, a slot extending longitudinally along the cylindrical body portion from the nose end to the rear flange, and opposing rails that extend longitudinally along edges of the cylindrical body portion formed by the slot; a needle disposed within the needle holder and comprising a venipuncture end extending forwardly from the nose end of the barrel and a discharge end extending rearwardly within the barrel cavity of the barrel; and a needle safety device comprising an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes the venipuncture end of the needle for selective use of the blood collection tube holder and a second extended position that positions the needle tip shield at least partially around the venipuncture end of the needle to protect a user from accidental needle sticks after use of the blood collection tube holder; inserting a blood collection tube into the blood collection tube holder; performing a blood draw on a patient; and activating the needle safety device to prevent unintended needle sticks or inadvertent contact with the venipuncture end of the needle upon completion of the blood draw.

Embodiment 52. The method of embodiment 51, wherein performing the blood draw on a patient comprises inserting the venipuncture end of the needle into the patient, collecting blood from the patient in the blood collection tube, and removing the venipuncture end of the needle from the patient.

Embodiment 53. The method of embodiment 52, further comprising: removing the blood collection tube from the blood collection tube holder.

Embodiment 54. The method of embodiment 53, wherein preventing unintended needle sticks or inadvertent contact with the venipuncture end of the needle is accomplished by sliding the needle safety device from a first retracted position to a second extended position to cause the needle tip shield of the needle safety device to circumferentially surround the venipuncture end of the needle and retaining the needle safety device in the second extended position by engagement of one or more slide stops disposed on the rails of the barrel with one or more ribs disposed in the channels of the needle safety device.

In the foregoing specification, the concepts have been described with reference to specific embodiments. However, those of ordinary skill in the art appreciate that various modifications and changes can be made without departing from the scope of the invention as set forth in the claims below. Other examples that occur to those skilled in the art are intended to be within the scope of the claims if they have structural elements that do not differ from the literal language of the claims, or if they include equivalent structural elements with insubstantial differences from the literal languages of the claims. Accordingly, the specification and figures are to be regarded in an illustrative rather than in a restrictive sense, and all such modifications are intended to be included within the scope of the invention.

Benefits, other advantages, and solutions to problems have been described above with respect to one or more specific embodiments. After reading the specification, those of ordinary skill in the art will appreciate that certain features that are, for clarity, described herein in the context of separate embodiments, may also be provided in combination in a single embodiment. Conversely, various features that are, for brevity, described in the context of a single embodiment, may also be provided separately or in any subcombination. Further, references to values stated in ranges include each and every value within that range and include sub-ranges that overlap from one preferred range to another preferred range.

What is claimed is:

1. A medical device, comprising:
a unitarily molded barrel comprising a cylindrical body portion that defines a barrel cavity, a nose end extending from the cylindrical body portion, a rear flange disposed around a rear end of the barrel cavity, a slot extending longitudinally along the cylindrical body portion from the nose end to the rear flange, and opposing rails that extend longitudinally along edges of the cylindrical body portion formed by the slot;
a needle coupled to the barrel and comprising a venipuncture end extending forwardly from the nose end of the barrel and a fluid discharge end extending rearwardly within the barrel cavity of the barrel; and
a needle safety device comprising an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes the venipuncture end of the needle for selective use of the medical device and a second extended position that positions the needle tip shield at least partially around the venipuncture end of the needle to protect a user from accidental needle sticks after use of the medical device.

2. The medical device of claim 1, wherein the nose end comprises an integral needle holder, and wherein the needle is secured within a needle holder via a friction or an interference fit.

3. The medical device of claim 1, wherein the nose end comprises an integral needle holder, and wherein the needle is secured within the needle holder via a medical grade adhesive.

4. The medical device of claim 1, further comprising: a needle holder threadably attached to the barrel to secure the needle to the barrel.

5. The medical device of claim 1, wherein the rails extend in a parallel manner along the cylindrical body portion along which the needle safety device can slide.

6. The medical device of claim 5, wherein the rails extend to and are integral with the rear flange to provide structural integrity to the cylindrical body portion of the barrel.

7. The medical device of claim 1, wherein the channels are formed in the main body portion of the needle safety device such that an upper portion of the main body portion extends over the rails of the barrel, and such that a lower portion of the main body portion extends below the rails of the barrel.

8. The medical device of claim 7, wherein the lower portion of the main body portion extends into radiused undercut portions formed on an inside of the cylindrical body portion of the barrel.

9. The medical device of claim 8, wherein the lower portion of the main body portion forms a portion of the barrel when the medical device is in use, and wherein the portion of the barrel formed by the lower portion of the main body portion of the needle safety device is selectively slidable after use of the medical device.

10. The medical device of claim 1, wherein extended portions of the activation surface of the needle safety device overlap the channels to lock the rails of the barrel within the channels.

11. The medical device of claim 1, wherein each of the rails comprise a rear stop and a slide stop disposed forwardly from the rear stop.

12. The medical device of claim 11, wherein the rear stops and slide stops extend inwardly from the rails.

13. The medical device of claim 12, wherein the rear stops are configured to engage ribs on the needle safety device to retain the needle safety device in the first retracted position to expose the venipuncture end of the needle for selective use of the medical device.

14. The medical device of claim 13, wherein the slide stops are configured to engage the ribs on the needle safety device and retain the needle safety device in the second extended position that is forward of the first retracted position to cover up the venipuncture end of the needle after use of the medical device to protect a user from accidental needle sticks after use of the medical device.

15. The medical device of claim 1, wherein the barrel comprises two opposing slots disposed on opposite sides of the barrel and extending longitudinally along the cylindrical body portion from the nose end to the rear flange, wherein each of the two slots comprise opposing rails that extend longitudinally along edges of the cylindrical body portion formed by each of the two slots.

16. The medical device of claim 15, wherein the needle safety device comprises a dual arm needle safety device that is slidably engaged with the two opposing slots disposed on opposite sides of the barrel.

17. The medical device of claim 16, wherein needle safety device comprises a plurality of main body portions extending from the needle tip shield and a pair of channels disposed along and formed in each of the main body portions, wherein the rails of each of the two opposing slots disposed on opposite sides of the barrel are received within the channels of the needle safety device to provide a slidable interface between the needle safety device and the barrel between the first retracted position and the second extended position.

18. A blood collection tube holder, comprising:
a unitarily molded barrel comprising a cylindrical body portion that defines a barrel cavity, a nose end extending from the cylindrical body portion, a needle holder, a rear flange disposed around a rear end of the barrel cavity, a slot extending longitudinally along the cylindrical body portion from the nose end to the rear flange, and opposing rails that extend longitudinally along edges of the cylindrical body portion formed by the slot; and
a needle safety device comprising an activation surface, a main body portion, a needle tip shield, and a pair of channels disposed along and formed in main body portion, wherein the rails of the barrel are received within channels of the needle safety device to provide a slidable interface between the needle safety device and the barrel between a first retracted position that exposes a venipuncture end of a needle for selective use of the blood collection tube holder and a second extended position that positions the needle tip shield at least partially around the venipuncture end of the needle to protect a user from accidental needle sticks after use of the blood collection tube holder.

19. The blood collection tube holder of claim 18, wherein the needle safety device is formed from a clear medical grade polymeric material that functions as a window to inspect the barrel cavity for blood flashback or a flow of fluid therethrough.

20. The blood collection tube holder of claim 19, further comprising: a needle disposed within the needle holder and comprising a venipuncture end extending forwardly from the nose end of the barrel and a fluid discharge end extending rearwardly within the barrel cavity of the barrel.

21. The blood collection tube holder of claim 18, wherein the rails extend to and are integral with the rear flange to provide structural integrity to the cylindrical body portion of the barrel.

22. The blood collection tube holder of claim 18, wherein the barrel comprises two opposing slots disposed on opposite sides of the barrel and extending longitudinally along the cylindrical body portion from the nose end to the rear flange, wherein each of the two slots comprise opposing rails that extend longitudinally along edges of the cylindrical body portion formed by each of the two slots, and wherein the needle safety device comprises a dual arm needle safety device that is slidably engaged with the two opposing slots disposed on opposite sides of the barrel.
